(19) United States
(12) Patent Application Publication
(10) Pub. No.: US 2020/0191780 A1
(43) Pub. Date: Jun. 18, 2020

(54) Nanocone Structure Composite Material for Capturing Cancer Cells, Preparation Method Therefor and Use Thereof

(71) Applicants: South China University of Technology, Guangzhou City, GD (CN); Guangdong University of Technology, Guangzhou City, Panyu District (CN)

(72) Inventors: Chengyun Ning, Guangzhou City (CN); Zhengao Wang, Guangzhou City (CN); Peng Yu, Guangzhou City (CN); Shiqian Hu, Guangzhou City (CN); Tiantian Yao, Guangzhou City (CN); Wen Luo, Guangzhou City (CN); Guoxin Tan, Guangzhou City, Panyu District (CN)

(21) Appl. No.: 16/616,627

(22) PCT Filed: Nov. 21, 2017
(86) PCT No.: PCT/CN2017/112178

§ 371 Date: Nov. 25, 2019

(30) Foreign Application Priority Data
May 25, 2017 (CN) 201710378368.5

(51) Int. Cl. G01N 33/543; C12N 5/09

(52) U.S. Cl. CPC G01N 33/54346; C12N 5/0693

(57) ABSTRACT

Disclosed are a nanocone structure composite material for capturing cancer cells, a preparation method therefor and the use thereof, which belong to the technical field of medical biomaterials. The method comprises: firstly, electrodepositing a chlorine-doped polypyrrole on the surface of a conductive substrate by using chronoamperometry; then, using chronopotentiometry and selecting a three-electrode mode with a conductive metal as a counter electrode, the conductive substrate deposited with the polypyrrole as a working electrode, and a buffer solution containing pyrrole and biotin as an electrolyte to deposit a nanocone structure polypyrrole/biotin material onto the working electrode; and finally, subjecting the working electrode deposited with the nanocone structure polypyrrole/biotin material to an activation treatment, placing the working electrode in a streptavidin solution for culturing, subjecting the working electrode to a grafting reaction with an antibody, and culturing the working electrode in a BSA solution to obtain a nanocone structure composite material. The method is simple and has low cost; and the nanocone structure in the composite material is stable and can better capture cancer cells and non-destructively release the cancer cells.

### TECHNICAL FIELD

The present invention belongs to the technical field of medical biomaterials, and relates to a nanocone structure composite material and a preparation method therefor, wherein the nanocone structure composite material is used for rapidly capturing cancer cells and non-destructively releasing the cells.

### BACKGROUND ART

Cancer cell separation is of a great significance in the study of basic biology, the development of clinical diagnosis, and treatment methods. At present, an antibody-antigen specific binding dependent technique for separating and purifying cancer cells by identifying a marker on the surface of a target cell membrane is developed. Compared to traditional benchtop methods, current platform-based technologies have the advantage of enhancing cell thawing and increasing the purity and captured amount of target cells. Although previous studies have focused on enhancing capture rate and sensitivity, there is also a lack of non-destructive release of cells and rapid capture of cells.

Nanostructured materials have very good performances and effects in cancer cell capture. Researchers have prepared a material for capturing and releasing cancer cells using an AAO template method; however, a process of removing the template involved in this method is realized by means of alkaline etching, which has an impact on the activity of biomolecules on the surface of the material; furthermore, the process is relatively complicated, and the prepared nanocone structure easily falls.

By utilizing the reversible doping characteristics and electrical activity of a polypyrrole, the present invention constructs a biotin-doped conductive polypyrrole platform by means of a dopant, with the platform being used for the capture and non-destructive release of EpCAM-positive cancer cells. The dopant can regulate and control the microstructure of the conductive polymer, which provides a possibility for preparing various nanostructures in a convenient, rapid and environmentally friendly manner. The present invention constructs a nanocone structure composite material using an electrochemical template-free method, with a simple process, no pollution, a good material stability, a high capture rate, and non-destructive release of cancer cells, thereby solving the defects and deficiencies present in the prior art.

### SUMMARY OF THE INVENTION

In order to overcome the defects and deficiencies of the prior art, an object of the present invention is to provide a method for preparing a nanocone structure composite material, i.e. a method for preparing a conductive polypyrrole/biotin nanocone structure composite material based on a conductive base. In the present invention, a nanocone structure composite material for capturing and releasing cancer cells is obtained by doping biotin into a polypyrrole by means of an electrochemical method to prepare a composite material having a nanocone structure, and then using a Biotin-Avidin-System (BAS) to graft an EpCAM antibody to the surface of the nanocone structure. The polypyrrole nanocone platform grafted with the EpCAM antibody of the present invention has a function of specific adhesion to EpCAM antibody-positive cells such as human colon cancer HCT-116 and human breast cancer cell MCF7, but has a poor adhesion to EpCAM antibody-negative cells such as cervical cancer cell Hela cells.

Another object of the present invention is to provide a nanocone structure composite material obtained by means of the above-mentioned method.

Still another object of the present invention is to provide the use of the above-mentioned nanocone structure composite material. The nanocone structure composite material is used for capturing and non-destructively releasing cancer cells, preferably EpCAM antibody-positive cells.

In order to achieve the objects of the present invention, the technical solutions used in the present invention are as follows:

a method for preparing a nanocone structure composite material, comprising the following steps:

(1) electrodeposition of a chlorine-doped polypyrrole onto the surface of a conductive substrate, wherein

a three-electrode mode is selected, with a conductive metal as a counter electrode, the conductive substrate as a working electrode and a solution containing pyrrole and chloride ions as an electrolyte solution, and chronoamperometry is used to control the electrochemical reaction to deposit the chlorine-doped polypyrrole onto the surface of the conductive substrate;

(2) deposition of a nanocone structure polypyrrole/biotin material onto the surface of a working electrode, wherein

a three-electrode mode is selected, with a conductive metal as a counter electrode, the conductive substrate deposited with the chlorine-doped polypyrrole, which is prepared in step (1), as the working electrode, and a buffer solution containing pyrrole and biotin as an electrolyte, and chronopotentiometry is used to control the electrochemical reaction to deposit the nanocone structure polypyrrole/biotin material onto the working electrode; and

(3) EpCAM antibody grafting, wherein

the working electrode deposited with the nanocone structure polypyrrole/biotin material in step (2) is placed in an aqueous solution of EDC and NHS for an activation treatment, then placed in a streptavidin solution for culturing, then subjected to a grafting reaction with a biotin-modified EpCAM antibody, and cultured in a BSA solution for a period of time to obtain an EpCAM antibody-grafted nanocone structure composite material.

The source of the chloride ions in step (1) is hydrochloric acid or potassium chloride, preferably hydrochloric acid.

The conductive metal in steps (1) and (2) is a platinum electrode or a copper electrode, preferably a copper electrode.

In step (1), the concentration of the chloride ions in the electrolyte solution is 0.1-0.3 mol/L, and the concentration of the pyrrole is 0.1-0.3 mol/L; and the time of the electrochemical reaction in step (1) is 10-50 s.

In step (1), the voltage of the electrochemical reaction is 0.7-1.2 V, preferably 0.8 V; and the conductive substrate is made of titanium, conductive glass, etc.

In step (1), the optimum concentration of the chloride ions is 0.25 mol/L, the optimum concentration of the pyrrole is 0.2 mol/L, and the optimum reaction time is 20 seconds.

The pH of the buffer solution in step (2) is 6.8-7.2, and the current of the electrochemical reaction in step (2) is 0.5-2.0 mA/cm²; and

the time of the electrochemical reaction in step (2) is 10-50 min.

In step (2), the concentration of the pyrrole is 0.1-0.3 mol/L, and the concentration of the biotin is 0.05-0.2 mol/L.

In step (2), the optimum concentration of the pyrrole is 0.2 mol/L, the optimum concentration of the biotin is 0.1 mol/L, and the optimal reaction time is 40 min.

The concentration of EDC in the aqueous solution of EDC and NHS in step (3) is 0.005-0.015 g/mL and the concentration of NHS is 0.005-0.015 g/mL; and the temperature of the activation treatment is normal temperature, and the time of the activation treatment is 30-60 min; the biotin-modified EpCAM antibody is purchased from R&D Systems, under product name: human EpCAM/TROP-1 biotinylated antibody; the time of the grafting reaction is 10-20 h, and the temperature of the grafting reaction is 4° C.−8° C.; the concentration of the aqueous streptavidin solution is 15-40 μg/mL, and the time of the culturing is 40-60 min; and the mass concentration of the BSA solution is 0.5%-1.5%, and the period of time is 40-60 min.

The nanocone structure composite material is prepared by means of the above-mentioned method. The nanocone structure composite material comprises a conductive substrate, a polypyrrole, biotin, and an antibody.

The nanocone structure composite material is used for the specific capture of cancer cells.

Compared with the prior art, the present invention has the following prominent advantages:

(1) in the present invention a conductive polypyrrole/biotin nanocone structure is constructed using a conductive substrate as a base by means of a non-polluting, fast and controllable electrochemical method, which realizes biotin doping to the polypyrrole;

(2) the nanocone structure polypyrrole/biotin composite material with a conductive substrate as a base, as constructed by means of an electrochemical template-free method, is simple in process and has cost and can be prepared and produced on a large scale; and the nanocone structure in the composite material prepared by the present application is stable; and

(3) the nanocone structure composite material of the present invention (an EpCAM antibody grafted on the surface of the conductive polypyrrole/biotin nanocone with a conductive substrate as a base) specifically captures cancer cells and non-destructively releases the cells.

### BRIEF DESCRIPTION OF THE DRAWINGS

FIG. 1 is an SEM image of a nanocone structure polypyrrole/biotin composite material (no antibody grafted) prepared in Example 1;

FIG. 2 is a cyclic voltammetry curve of the nanocone structure polypyrrole/biotin composite material (no antibody grafted) prepared in Example 1;

FIG. 3 is laser confocal microscope images of a nanocone structure composite material (grafted with an antibody) prepared in Example 5 for specifically capturing cancer cells, wherein a1 and a2 correspond to HCT116 cells, b1 and b2 correspond to MCF7 cells, and c1 and c2 correspond to HeLa cells, with a1 and a2, b1 and b2, and c1 and c2 respectively being different magnification factors; and

FIG. 4 is laser confocal microscope images of the capture of MCF7 cancer cells by the nanocone structure composite material (EpCAM antibody functionalized) prepared in Example 5 (a) and the release of the MCF7 cancer cells under weak potential, short-term stimulation (b).

### DETAILED DESCRIPTION OF EMBODIMENTS

The present invention will be further described in detail below in conjunction with embodiments and accompanying drawings, but this does not limit the implementation of the present invention.

### Example 1

(1) A sheet-like conductive titanium substrate has a specification of 10×10×1 mm³, and the substrate is super-cleaned respectively with deionized water, 99.7% anhydrous ethanol and 99.5% acetone, each for 20 minutes;

(2) a three-electrode mode is selected, with a conductive substrate as a working electrode, a copper plate as a counter electrode, a saturated calomel electrode as a reference electrode, and an electrolyte solution having a pyrrole concentration of 0.2 mol/L and a hydrochloric acid concentration of 0.25 mol/L, chronoamperometry is used to control the electrochemical reaction for a reaction time of 20 seconds, with a reaction potential (with respect to the reference electrode) of 0.8 V, to deposit a dense, homogeneous black polypyrrole on the titanium electrode, and the titanium electrode is soaked in deionized water to remove unreacted pyrrole and hydrochloric acid from the surface to obtain a titanium electrode deposited with polypyrrole; and

(3) a three-electrode mode is selected, with the titanium electrode deposited with polypyrrole as a working electrode, a copper plate as a counter electrode, a saturated calomel electrode as a reference electrode, and a buffer solution of pyrrole and biotin (the pH of the solution is 6.8, PBS) as an electrolyte solution, in which the concentration of pyrrole is 0.2 mol/L and the concentration of biotin is 0.1 mol/L, and chronopotentiometry is used to control the electrochemical reaction for a reaction time of 40 minutes, with a reaction current of 1.5 mA, to deposit a nanocone structure polypyrrole/biotin complex onto the surface of the working electrode to obtain a nanostructured polypyrrole/biotin composite (no antibody grafted), i.e. a polypyrrole/biotin material working electrode deposited with a nanocone structure.

The SEM image of the nanocone structure polypyrrole/biotin composite material (no antibody grafted) of this example is as shown in FIG. 1. As can be seen from FIG. 1, a high-density nanocone structure is deposited on the surface of the titanium electrode and is grown perpendicularly to the surface; and the nanocone structure has an apex outer diameter of 75 nm and a vertical height of 500 nm.

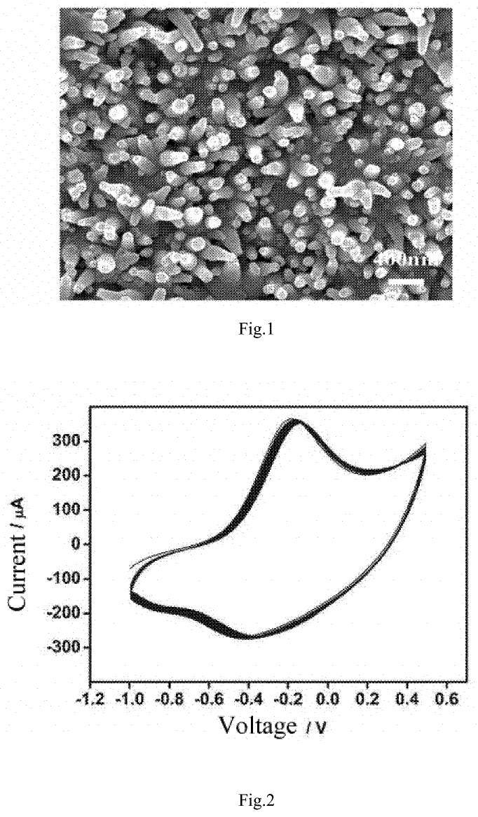

The cyclic voltammetry curve of the nanocone structure polypyrrole/biotin composite material (no antibody grafted) of this example is as shown in FIG. 2. The test conditions are: PBS as an electrolyte, the nanocone structure polypyrrole/biotin composite material (i.e. the working electrode deposited with the nanocone structure polypyrrole/biotin material) prepared in Example 1 as a working electrode, an electrochemical workstation to record a cyclic voltammetry curve, a sweep speed of 25 mV/s, and 10 cycles of scanning. The results show that the nanocone structure polypyrrole/biotin composite material has better redox properties.

### Example 2

(1) A sheet-like conductive substrate (a conductive glass) has a specification of 10×10×1 mm³, and the substrate is super-cleaned respectively with deionized water, 99.7% anhydrous ethanol and 99.5% acetone, each for 20 minutes;

(2) a three-electrode mode is selected, with a conductive substrate as a working electrode, a copper plate as a counter electrode, a saturated calomel electrode as a reference electrode, and an electrolyte solution having a pyrrole concentration of 0.2 mol/L and a hydrochloric acid concentration of 0.25 mol/L, chronoamperometry is used to control the electrochemical reaction for a reaction time of 20 seconds, with a reaction potential (with respect to the reference electrode) of 0.8 V, to deposit a dense, homogeneous black polypyrrole on the conductive glass electrode, and the conductive glass electrode is soaked in deionized water to remove unreacted pyrrole and hydrochloric acid from the surface to obtain a conductive glass electrode deposited with polypyrrole; and

(3) a three-electrode mode is selected, with the conductive glass electrode deposited with polypyrrole as a working electrode, a copper plate as a counter electrode, a saturated calomel electrode as a reference electrode, and a buffer solution of pyrrole and biotin (the pH of the solution is 7.2, PBS) as an electrolyte solution, in which the concentration of pyrrole is 0.2 mol/L and the concentration of biotin is 0.05 mol/L, and chronopotentiometry is used to control an electrochemical reaction for a reaction time of 40 minutes, with a reaction current of 1.5 mA, to deposit a nanocone structure polypyrrole/biotin complex onto the surface of the working electrode to obtain a nanostructured polypyrrole/biotin composite (no antibody grafted). The composite material structure prepared in this example is similar to that of Example 1, and the electrochemical performance thereof is also similar to that of Example 1.

### Example 3

(1) A sheet-like conductive titanium substrate has a specification of 10×10×1 mm³, and the substrate is super-cleaned respectively with deionized water, 99.7% anhydrous ethanol and 99.5% acetone, each for 20 minutes;

(2) a three-electrode mode is selected, with a conductive substrate as a working electrode, a copper plate as a counter electrode, a saturated calomel electrode as a reference electrode, and an electrolyte solution having a pyrrole concentration of 0.2 mol/L and a hydrochloric acid concentration of 0.25 mol/L, chronoamperometry is used to control the electrochemical reaction for a reaction time of 20 seconds, with a reaction potential (with respect to the reference electrode) of 0.8 V, to deposit a dense, homogeneous black polypyrrole on the titanium electrode, and the titanium electrode is soaked in deionized water to remove unreacted pyrrole and hydrochloric acid from the surface to obtain a titanium electrode deposited with polypyrrole; and

(3) a three-electrode mode is selected, with the titanium electrode deposited with polypyrrole as a working electrode, a copper plate as a counter electrode, a saturated calomel electrode as a reference electrode, and a buffer solution of pyrrole and biotin (the pH of the solution is 6.8, PBS) as an electrolyte solution, in which the concentration of pyrrole is 0.2 mol/L and the concentration of biotin is 0.1 mol/L, and chronopotentiometry is used to control an electrochemical reaction for a reaction time of 40 minutes, with a reaction current of 0.9 mA, to deposit a nanocone structure polypyrrole/biotin complex onto the surface of the working electrode to obtain a nanostructured polypyrrole/biotin composite (no antibody grafted). The composite material structure prepared in this example is similar to that of Example 1, and the electrochemical performance thereof is also similar to that of Example 1.

### Example 4

(1) A sheet-like conductive titanium substrate has a specification of 10×10×1 mm³, and the substrate is super-cleaned respectively with deionized water, 99.7% anhydrous ethanol and 99.5% acetone, each for 20 minutes;

(2) a three-electrode mode is selected, with a conductive substrate as a working electrode, a copper plate as a counter electrode, a saturated calomel electrode as a reference electrode, and an electrolyte solution having a pyrrole concentration of 0.2 mol/L and a potassium chloride concentration of 0.2 mol/L, chronoamperometry is used to control an electrochemical reaction for a reaction time of 20 seconds, with a reaction potential (with respect to the reference electrode) of 0.8 V, to deposit a dense, homogeneous black polypyrrole on the titanium electrode, and the titanium electrode is soaked in deionized water to remove unreacted pyrrole and potassium chloride from the surface to obtain a titanium electrode deposited with polypyrrole; and

(3) a three-electrode mode is selected, with the titanium electrode deposited with polypyrrole as a working electrode, a copper plate as a counter electrode, a saturated calomel electrode as a reference electrode, and a buffer solution of pyrrole and biotin (the pH of the solution is 6.8, PBS) as an electrolyte solution, in which the concentration of pyrrole is 0.2 mol/L and the concentration of biotin is 0.1 mol/L, and chronopotentiometry is used to control an electrochemical reaction for a reaction time of 40 minutes, with a reaction current of 2.0 mA, to deposit a nanocone structure polypyrrole/biotin complex onto the surface of the working electrode to obtain a nanostructured polypyrrole/biotin composite (no antibody grafted). The composite material structure prepared in this example is similar to that of Example 1, and the electrochemical performance thereof is also similar to that of Example 1.

### Example 5

The working electrode deposited with the nanocone structure polypyrrole/biotin material as prepared in Example 1 is soaked in 10 mL of an aqueous solution of EDC (0.095 g) and NHS (0.061 g), undergoes an activation treatment for 45 minutes at normal temperature, and is rinsed 3 times with ultrapure water, the nanocone structure polypyrrole/biotin material on the working electrode is activated, and the working electrode is soaked in 50 μL of an aqueous solution of streptavidin (20 μg/mL) for 1 hour (normal temperature), taken out and rinsed 3 times with ultrapure water; the working electrode is re-soaked in a solution of biotin-modified EpCAM antibody (human EpCAM/TROP-1 biotin antibody, R&D Systems) (10 μg/mL, lx PBS as a solvent (1×PBS refers to the concentration used during cell culture)), cultured for 12 hours in a 4° C. environment, washed 3 times with a PBS solution (standard PBS used during cell culture), then soaked in a BSA protein solution (1 wt %, 1×PBS as a solvent) for 1 hour of culture at room temperature for reducing non-specific binding, and finally washed three times with PBS to obtain a nanocone structure composite material.

The nanocone structure composite material prepared in Example 5 is tested for the effect of capturing and releasing cancer cells:

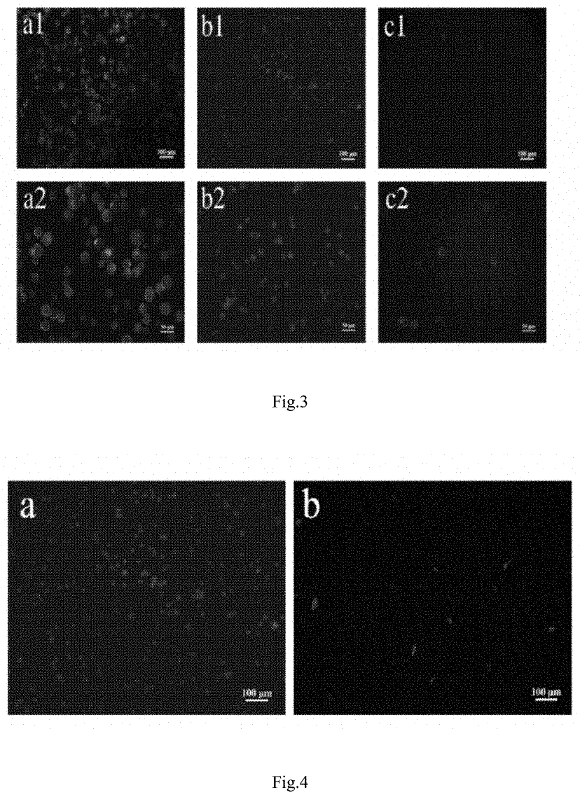

(A) the nanocone structure composite material prepared in Example 5 is used for specifically capturing cancer cells, and the results thereof (laser confocal microscope image) are as shown in FIG. 3, wherein a1 and a2 correspond to HCT116 cells, b1 and b2 correspond to MCF7 cells, and c1 and c2 correspond to HeLa cells, with a1 and a2, b1 and b2, and c1 and c2 respectively being different magnification factors.

HCT116 and MCF7 are respectively human colon cancer cells and human breast cancer cells, and can specifically recognize EpCAM antibodies; and Hela cells are cervical cancer cells and cannot specifically recognize EpCAM antibodies. After co-culturing the nanocone composite material and cancer cells at a concentration of 2×10⁵/mL for 15 minutes, HCT116 cells (FIG. 3(a)) and MCF7 cells (FIG. 3(b)) are adhered to the surface of the material in large amounts, with the cell density of the HCT116 cells on the surface of the material being 260±25/mm² and the cell density of the MCF7 cells on the surface of the material being 252±18/mm². In contrast, HeLa cells (FIG. 3(c)) are difficult to adhere to the surface of the EpCAM antibody-functionalized polypyrrole nanocone structure in a short time, and the cell density on the surface of the material is only 41±9/mm².

(B) The nanocone structure composite material prepared in Example 5 is tested for cancer cell release.

Human colon cancer cells HCT-116, human breast cancer cells MCF7 and cervical cancer cells Hela are cultured, with the cell medium being an α-MEM medium of fetal bovine serum (FBS) having a volume fraction of 10%. The HCT-116, MCF-7 and HeLa cells are cultured in a constant temperature incubator at 37° C. and with 5% CO₂, and the medium is changed once every 2 days depending on solution conditions. When the cell spread density reaches 70%-80%, the cells are passaged or inoculated onto the surface of the material, with the cell inoculation density being 2×10⁵/mL. For the cell inoculation, the sample is closely adhered to the bottom of a perforated 48-well plate. Each hole is designed as a three-electrode electrolytic cell, with the nanocone structure composite material as a working electrode, a platinum wire as a counter electrode, and Ag/AgCl as a reference electrode. The inoculated cells are subjected to Actin skeleton staining and then observed by means of laser confocal microscopy. The laser confocal microscope image of capturing MCF7 cancer cells by the nanocone structure composite material (EpCAM antibody functionalized) prepared in Example 5 is as shown in FIG. 4(a).

An electrochemical workstation is used to apply a voltage to an electrolytic cell for culturing the cells. The voltage is 0.8 V and the electrical stimulation time is 15 seconds. The laser confocal microscope image of the release of MCF7 cancer cells under short-term, weak potential stimulation is as shown in FIG. 4(b). It can be seen from the comparison between (a) and (b) that after the nanocone structure composite material captures the cells, the MCF7 cancer cells on the surface of the material are substantially released under the short-term, weak potential stimulation.

1. A method for preparing a nanocone structure composite material, characterized by comprising the following steps:
(1) electrodeposition of a chlorine-doped polypyrrole onto the surface of a conductive substrate, wherein
a three-electrode mode is selected, with a conductive metal as a counter electrode, the conductive substrate as a working electrode and a solution containing pyrrole and chloride ions as an electrolyte solution, and chronoamperometry is used to control the electrochemical reaction to deposit the chlorine-doped polypyrrole onto the surface of the conductive substrate;
(2) deposition of a nanocone structure polypyrrole/biotin material onto the surface of a working electrode, wherein
a three-electrode mode is selected, with a conductive metal as a counter electrode, the conductive substrate deposited with the chlorine-doped polypyrrole, which is prepared in step (1), as the working electrode, and a buffer solution containing pyrrole and biotin as an electrolyte, and chronopotentiometry is used to control the electrochemical reaction to deposit the nanocone structure polypyrrole/biotin material onto the working electrode; and
(3) EpCAM antibody grafting, wherein
the working electrode deposited with the nanocone structure polypyrrole/biotin material in step (2) is placed in an aqueous solution of EDC and NHS for an activation treatment, then placed in a streptavidin solution for culturing, then subjected to a grafting reaction with a biotin-modified EpCAM antibody, and cultured in a BSA solution for a period of time to obtain an EpCAM antibody-grafted nanocone structure composite material.

2. The method for preparing a nanocone structure composite material according to claim 1, characterized in that the pH of the buffer solution in step (2) is 6.8-7.2, and the current of the electrochemical reaction in step (2) is 0.5-2.0 mA/cm².

3. The method for preparing a nanocone structure composite material according to claim 1, characterized in that the source of the chloride ions in step (1) is hydrochloric acid or potassium chloride; and
the conductive metal in steps (1) and (2) is a platinum electrode or a copper electrode.

4. The method for preparing a nanocone structure composite material according to claim 3, characterized in that the source of the chloride ions in step (1) is hydrochloric acid; and
the conductive metal in steps (1) and (2) is a copper electrode.

5. The method for preparing a nanocone structure composite material according to claim 1, characterized in that the time of the electrochemical reaction in step (1) is 10-50 s;
the voltage of the electrochemical reaction in step (1) is 0.7-1.2 V; and the time of the electrochemical reaction in step (2) is 10-50 min.

6. The method for preparing a nanocone structure composite material according to claim 1, characterized in that in step (1), the concentration of the chloride ions in the electrolyte solution is 0.1-0.3 mol/L, and the concentration of the pyrrole is 0.1-0.3 mol/L; and
in step (2), the concentration of the pyrrole is 0.1-0.3 mol/L, and the concentration of the biotin is 0.05-0.2 mol/L.

7. The method for preparing a nanocone structure composite material according to claim 1, characterized in that in step (3), the time of the grafting reaction is 10-20 h, and the temperature of the grafting reaction is 4° C.-8° C.; the temperature of the activation treatment is normal temperature, and the time of the activation treatment is 30-60 min; the time of the culturing is 40-60 min; and the period of time is 40-60 min.

8. The method for preparing a nanocone structure composite material according to claim 1, characterized in that the concentration of EDC in the aqueous solution of EDC and NHS in step (3) is 0.005-0.015 g/mL and the concentration of NHS is 0.005-0.015 g/mL; and the concentration of the aqueous streptavidin solution is 15-40 μg/mL, and the mass concentration of the BSA solution is 0.5%-1.5%.

9. A nanocone structure composite material obtained by means of the method of claim 1.

10. The use of the nanocone structure composite material according to claim 9, characterized in that the nanocone structure composite material is used for the specific capture of cancer cells.

11. A nanocone structure composite material obtained by means of the method of claim 2.

12. A nanocone structure composite material obtained by means of the method of claim 3.

13. A nanocone structure composite material obtained by means of the method of claim 4.

14. A nanocone structure composite material obtained by means of the method of claim 5.

15. A nanocone structure composite material obtained by means of the method of claim 6.

16. A nanocone structure composite material obtained by means of the method of claim 7.

17. A nanocone structure composite material obtained by means of the method of claim 8.
